(19) United States
(12) Patent Application Publication
(10) Pub. No.: US 2004/0158408 A1
(43) Pub. Date: Aug. 12, 2004

(54) Server-client network system for genotyping analysis and computer readable medium therefor

(76) Inventors: Seung-Hak Choi, Gyeonggi-do (KR); Yunsun Nam, Seoul (KR); Taejoon Kwon, Gyeonggi-do (KR); Tae Su Chung, Seoul (KR); Kwonmoo Lee, Cambridge, MA (US); Hoseung Lee, Gyeonggi-do (KR)

Correspondence Address: Soonja Bae, Cantor Colburn LLP, 55 Griffin Road South, Bloomfield, CT, 06002, US

(21) Appl. No.: 10/765,547

(22) Filed: Jan. 26, 2004

(30) Foreign Application Priority Data
Jan. 27, 2003 (KR) 2003-5199

(51) Int. Cl. G06F 19/00; G01N 33/48; G01N 33/50

(52) U.S. Cl. 702/20

(57) ABSTRACT

A server-client network system for genotyping analysis and a computer readable medium used therein are provided. The server-client network system includes a server storing databases required for genotyping analysis and providing the databases to a client and the client receiving the results of a test on the target sample using a biochip and performing a genotyping analysis on the target sample with reference to the databases provided from the server. The server-client network system improves the security of privacy information and guarantees the independency of analysis software.

### BACKGROUND OF THE INVENTION

This application claims the priority of Korean Patent Application No. 2003-5199, filed on Jan. 17, 2003, in the Korean Intellectual Property Office, the disclosure of which is incorporated herein in its entirety by reference.

1. Field of the Invention

The present invention relates to a server-client network system for genotyping analysis and a computer readable medium therefor.

2. Description of the Related Art

Advances in medical technology and increasing concerns about health have led to the appearance of e-healthcare systems that organically offer various healthcare services for disease prevention, diagnosis, examination, nursing, management, etc. over the Internet.

The concept of healthcare is rapidly changing from island care to wellness management. Island care implies the cure-centered healthcare provided at a particular place, for example, a hospital, after patients fall sick. Wellness management implies the home-centered health care provided through a network so as to diagnose and cure a patient at home or prevent the occurrence of diseases.

In particular, with recent rapid developments in biochip related technologies, a new e-healthcare concept based on genotyping diagnosis has been introduced, as disclosed in International Patent Publication No. 01/16860.

According to the disclosure in International Patent Publication No. 01/16860, only a server is allowed to analyze genotyping data. However, since genotyping analysis on a number of individuals is performed only in the server, there is a delicate problem in securing private information. Namely, when the results of a biochip test are provided to a client after being written into a database of the server, the private test information is highly likely to be exposed.

Unlike the above patent, when only clients are responsible for genotyping analysis, every client must be provided with a new version of user software whenever a new biochip is released. In other words, biochips that have an identical platform but different contents are treated as different ones, so that different versions of user software should also be provided with the biochips. Consequently, version management becomes complicated due to the need for various versions of analysis software. Moreover, the various versions of analysis software require continuous updating. As test data are accumulated more and more, the accuracy of genotyping may be improved by varying algorithms or parameters. However, varying algorithms or parameters for this purpose requires a full change of the software.

Therefore, there is a need for a novel server-client network system for biochip-based e-healthcare services, which protects privacy information and guarantees the independency of analysis software.

### SUMMARY OF THE INVENTION

The present invention provides a server-client network system for genotyping analysis that protects privacy information and guarantees the independency of analysis software.

The present invention also provides a computer readable medium for a client of the above server-client network system.

In one aspect of the present invention, there is provided a server-client network system for a genotyping analysis on a target sample, the server-client network system comprising: a server including databases required for the genotyping analysis and providing the databases to a client; and the client receiving the results of a biochip test on the target sample using a biochip and performing the genotyping analysis on the target sample with reference to the databases provided from the server.

The databases may include: a biochip identifier and layout database storing information on the identifier and layout of the biochip; an analysis algorithm database storing algorithms required for genotyping analysis; and a quality control criteria database.

In another aspect of the present invention, there is provided a computer readable medium for a server-client network system for genotyping analysis, the computer readable medium having computer executable instructions for a client to perform logical operations for genotyping analysis.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram of a server-client network system according to the present invention;

FIG. 2 is a block diagram of a server and a client that construct the server-client network system according to the present invention;

FIG. 3 shows an embodiment of a biochip identifier (ID) and layout database (DB) stored in the server;

FIG. 4 shows an embodiment of an analysis algorithm DB stored in the server;

FIG. 5 shows an embodiment of a quality control criteria DB stored in the server;

FIG. 6 is a flowchart illustrating genotyping analysis in a client engine; and

FIG. 7 is a flowchart illustrating the operation in FIG. 6 in detail.

### DETAILED DESCRIPTION OF THE INVENTION

Embodiments of a server-client network system and a computer readable medium used therein according to the present invention will be described in detail with reference to the appended drawings. The present invention may, however, be embodied in many different forms and should not be construed as being limited to the embodiments set forth herein; rather, these embodiments are provided so that this disclosure will be thorough and complete, and will fully convey the concept of the invention to those skilled in the art. The spirit and scope of the present invention are defined only by the claims. In the drawings and specification, like reference numerals are used to refer to like elements throughout.

The term “biochip” used throughout the specification embraces any chip of probes in a microarray, such as DNA chips, protein chips, etc. A DNA chip comprises a microarray of a number of nucleic acid probes, wherein a nucleic acid implies nucleoside or a nucleotide polymer or oligomer (also referred to as polynucleotide or oligonucleotide) including pyrimidine and purine. The pyrimidine includes cytosine (C), thymine (T), and uracile (U). The purine includes adenine (A) and guanine (G). Examples of DNA chips include cDNA chips using at least 500 bp or greater probes and oligonucleotide chips using 9-25 mer oligonucleotide probes. A protein chip comprises a microarray of tens to hundreds of protein or ligand probes.

The term of “target sample” used throughout the specification refers to a nucleic acid (oligonucleotide or polynucleotide of RNA or DNA) to be genotyped or a protein to be functionally identified. The term “probe” refers to a material used to hybridize to and detect the target sample.

In a server-client system according to the present invention, databases (DBs) required for genotyping analysis, for example, a biochip identifier (ID) and layout DB, a genotyping analysis algorithm DB, a quality control (QC) reference DB, etc, are provided to a client by the server. The client analyses the results of a biochip test on a target sample with reference to the DBs provided from the server and stores the test results.

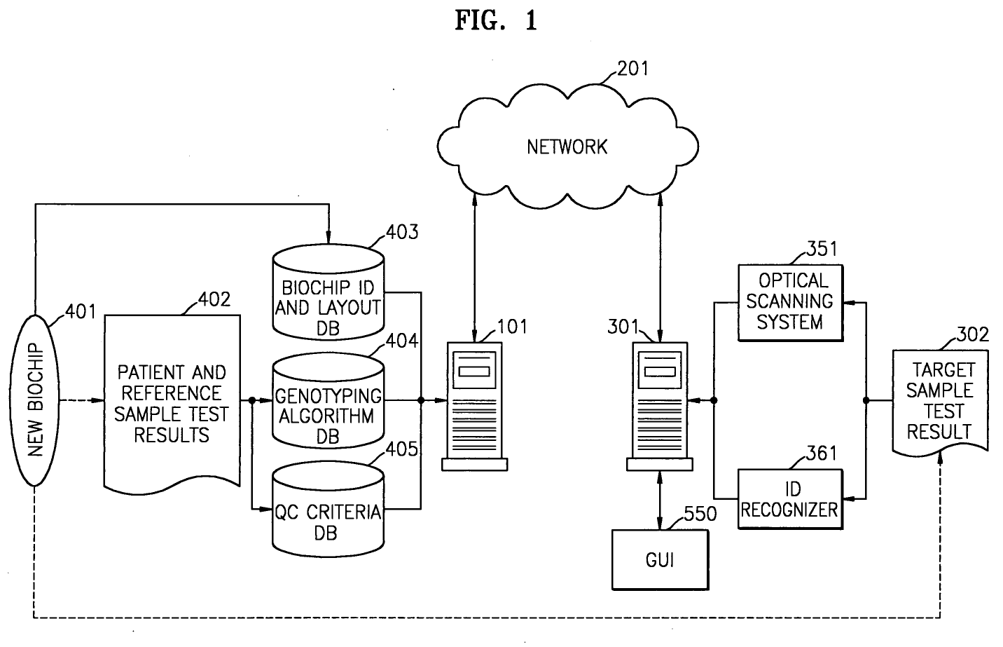

Referring to FIG. 1, which shows a server-client system according to the present invention, a server 101 and a client 301 are connected to each other via a network 201 to receive information from and/or to transmit information to the other for communication. The network 201 may be a wide area network (WAN), such as the Internet, or a local area network (LAN), such as the Ethernet.

A biochip ID and layout DB 403 for a newly introduced biochip 401 is stored in the server 101. After testing on a number of patient and reference samples using the biochip 401, a genotyping algorithm DB 404 and a QC criteria DB 405 are established using statistical data for the results of the test and then stored in the server 101.

The client 301 is connected with an optical scanning system 351 via which a target sample test result 302, i.e., the result of hybridization of a target sample to the biochip 401, is input and an ID recognizer 361 which recognizes the ID of the biochip 401 and inputs the detected ID to the client 301.

The client 301 discriminates and analyzes the genotype of the target sample using the target sample test result 302 input via the optical scanning system 351 and the biochip ID input via the ID recognizer 361 with reference to the biochip ID and layout DB 403, the genotyping algorithm DB 404, and the QC criteria DB 405 provided by the server 101. Optionally, the biochip ID and layout DB 403, the genotype analysis algorithm DB 404, and the QC criteria DB 405 stored in the server 101 may be replicated as mirror databases in the client 301.

The results of genotyping analysis are stored in the client 301. The results of genotyping analysis may be provided to a user by means of a graphical user interface (GUI) 550. The GUI 550 may provide a file search window, a list of results window, and/or an analysis result access window, etc.

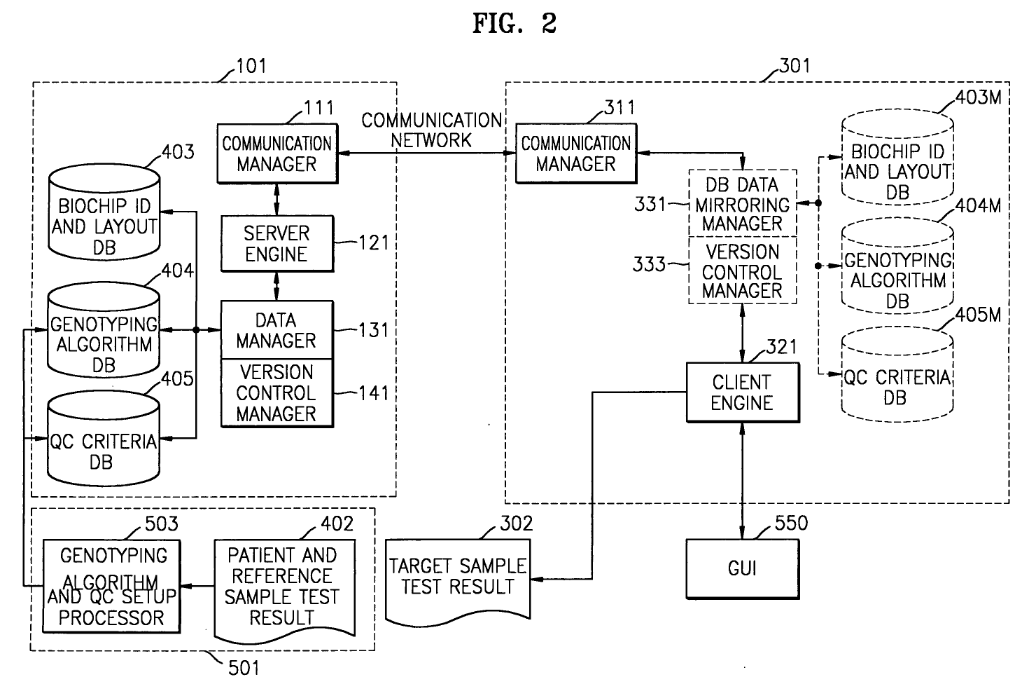

FIG. 2 is a block diagram of the server-client network system according to the present invention. A communication manager 111 installed in the server 101 and a communication manager 311 installed in the client 301 enable the server 101 and the client 301 to communicate with each other.

All functions of the server 101 are controlled by a server engine 121. A data manager 131 manages the biochip ID and layout DB 403, the genotyping algorithm DB 404, and the QC criteria DB 405 under the control of the server engine 121. A version control manager 141 manages versions of the biochip ID and layout DB 403, the genotype analysis algorithm DB 404, and the QC criteria DB 405 managed by the data manager 131.

In the server 101, parameters required for genotyping and diagnostic analysis and QC are calculated using several test results 402, which are obtained by the hybridization of patient and reference samples to the biochip 401, with reference to the genotyping algorithm DB 404 and the QC criteria DB 405 and then stored. This operation may be performed by a genotyping algorithm and QC setup processor 503. Collecting the test results 402 for the patient and reference samples and operating the genotyping algorithm and QC setup processor 503 may be performed in the server 101 itself or in a separate server 501 as illustrated in FIG. 2.

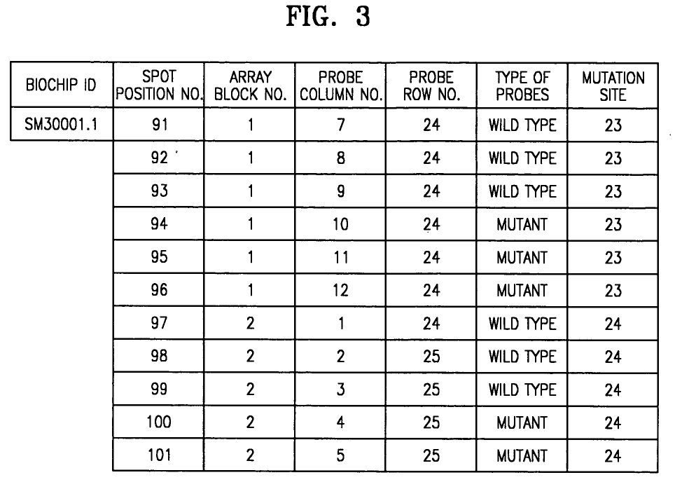

FIG. 3 shows an embodiment of the biochip ID and layout DB 403 stored in the server 101. Referring to FIG. 3, a biochip ID and layout DB for a biochip with an ID number, for example, SM30001.1, includes various kinds of mapping information, for example, spot position serial numbers, array block numbers, probe positions (in column and row), types of probes, and mutation sites corresponding to probes.

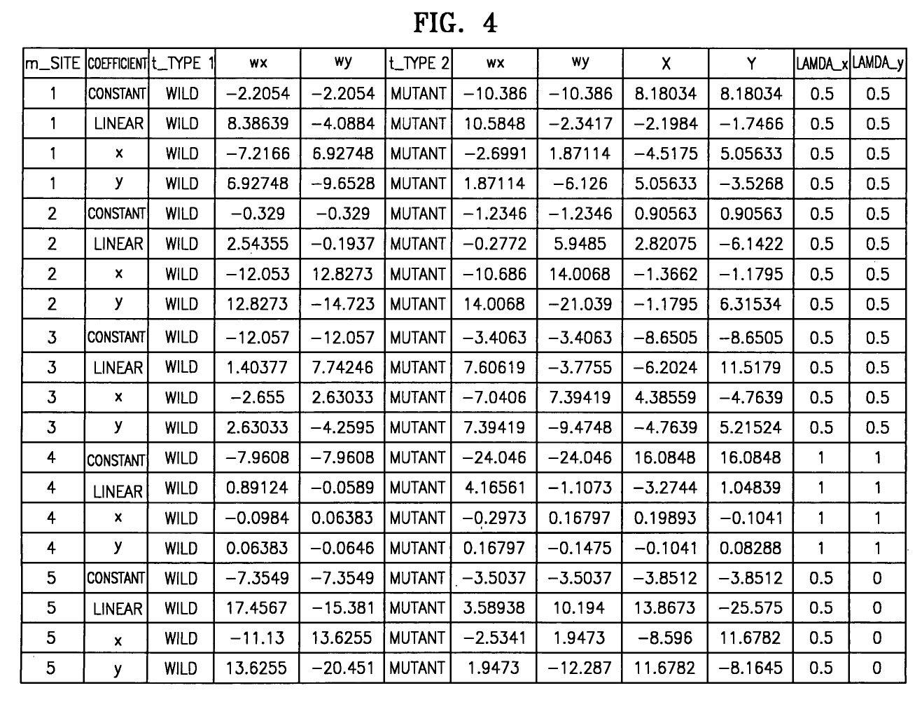

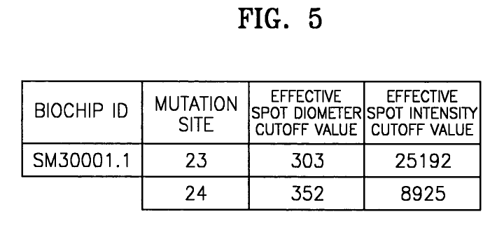

FIGS. 4 and 5 show embodiments of the genotyping algorithm DB 404 and the QC criteria DB 405 built up by the genotyping algorithm and QC setup processor 503, respectively. As shown in FIG. 4, the genotyping algorithm DB 404 may include algorithm types applied to each mutation site and corresponding parameters for the algorithm types. As shown in FIG. 5, the QC criteria DB 405 may include effective spot diameter cutoff values and effective spot intensity cutoff values.

Referring back to FIG. 2, all functions of the client 301 are managed by a client engine 321. The client engine 321 receives a target sample test result 302, which is the result of hybridization of the target sample to the biochip, via the optical scanning system 351 (see FIG. 1), pre-processes the received target sample test result 302, and performs genotype analysis using the pre-processed target sample test result and the biochip ID recognized by the ID recognizer 361 (see FIG. 1).

The client engine 321 includes a computer readable medium having computer executable instructions for logical steps including receiving from the server 101 the biochip ID and layout DB 403, the genotyping algorithm DB 404, and the QC criteria DB 405 required for genotyping analysis and performing genotyping analysis by analysing the results of the biochip test on the target sample with reference to the DBs 403, 404, and 405.

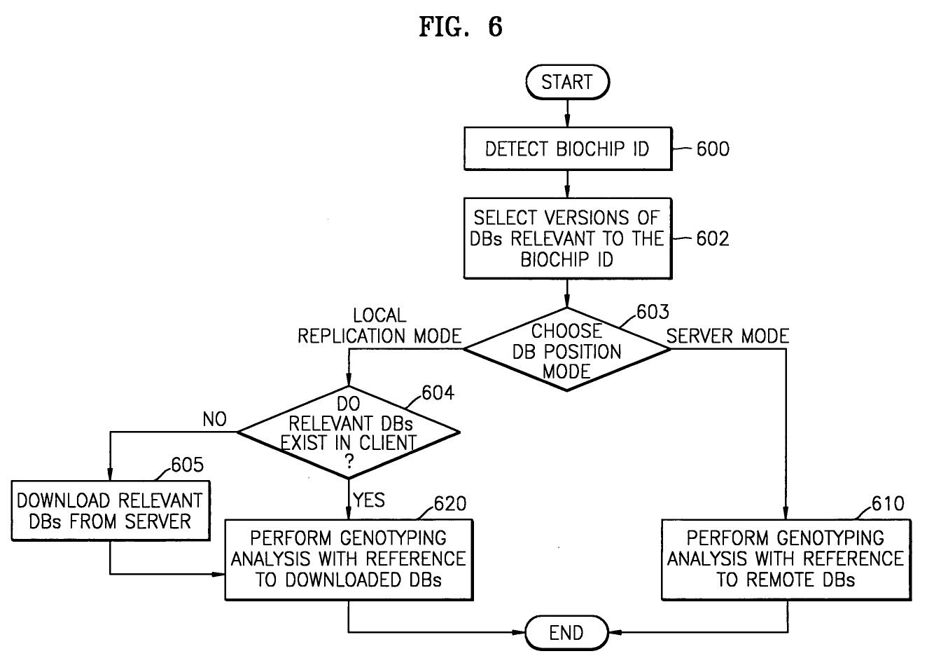

Genotyping analysis performed in the client engine 321 will now be described in detail with reference to FIG. 6. FIG. 6 is a flowchart of a genotyping analysis method performed in the client engine 321. Referring to FIG. 6, the client engine 321 detects the ID of the biochip (step 600). Versions of DBs relevant to the ID are selected (step 602), and a DB position mode is chosen (step 603). The DB position mode includes a local replication mode and a server mode. When the sever mode is chosen, the communication managers 311 and 111 communicate with each other to allow the client engine 321 to analyze genotypes with reference to the remote DBs 403, 404, and 405, which are stored in the server 101 (step 610).

When the local replication mode is chosen, the client engine 321 determines whether DBs relevant to the ID exist in the client 301 (step 604). If no relevant DB exists, the relevant DBs are downloaded from the server 101 via the communication managers 111 and 311 (step 605). A DB data mirroring manager 331 (see FIG. 2) and a version control manger 333 (see FIG. 2) manage the download of the relevant DBs. The client engine 321 analyzes genotypes with reference to local mirror databases 403M, 404M, and 405M (step 620).

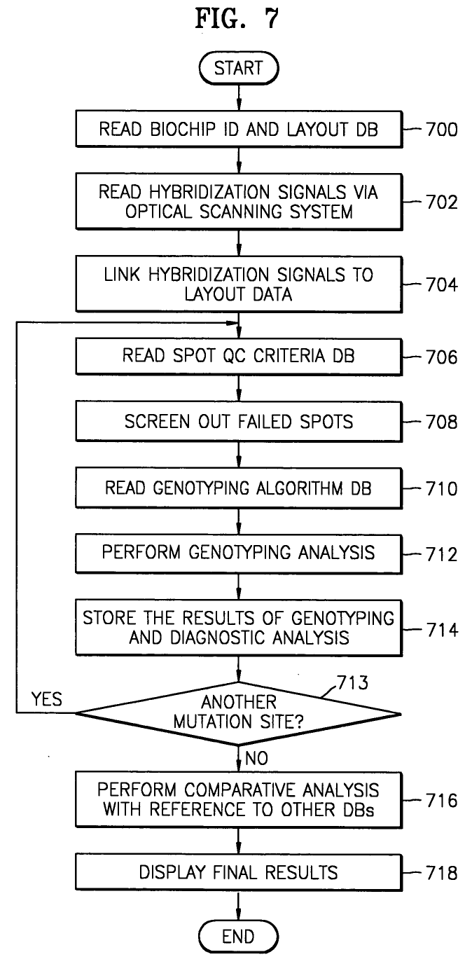

FIG. 7 is a flowchart of step 610 or 620 of genotyping analysis on the target sample. The biochip ID and layout DB 403 (403M) is read (step 700). The target sample test result, for example, the intensities of hybridization signals, input via the optical scanning system, are read (step 702). The target sample test result is linked to the layout data read from the biochip ID and layout DB 403 (403M) (step 704), the QC criteria DB 405 (405M), for example, a spot QC criteria DB, is read (step 706), and failed spots that do not satisfy the QC criteria are screened out (step 708). Next, the genotyping algorithm DB 404 (404M) is read, a genotyping analysis is performed with reference to the DBs, and the results of the genotyping analysis are stored in the client 321 (step 714). Next, it is determined whether another mutation site exists (step 713). If another mutation site is detected, steps 706 through 714 are repeated. Optionally, a comparative analysis with reference to another DB, for example, a mutant DB, a criteria DB, and/or a disease DB, may be further performed. The final results of the genotyping analysis are displayed (step 718).

While the present invention has been particularly shown and described with reference to exemplary embodiments thereof, it will be understood by those of ordinary skill in the art that various changes in form and details may be made therein without departing from the spirit and scope of the present invention as defined by the following claims.

As described above, a genotyping analysis using biochips can be more practically performed using a server-client network system according to the present invention. Parameters for genotyping analysis and QC are extracted from the results of a biochip test on patient and reference samples, and the client utilizes the parameters in genotyping analysis. As the test results are accumulated, new parameters for the accumulated test results can be released from the server to the client to raise genotyping efficiency.

The server-client network system for genotyping analysis according to the present invention improves security since only the client is allowed to store the results of a biochip test and genotype the test results with reference to relevant DBs provided from the server.

The server-client network system for genotyping analysis according to the present invention is convenient to use. Up-to-date information required for genotyping analysis is managed by the server, and separate versions of software are unnecessary for chips that have the same platform but different contents. In addition, a version of software can be automatically matched with the up-to-date information based on the chip ID. The cumulated results of genotyping analysis can be utilized to improve test accuracy. A user can more conveniently utilize the server-client network system via a graphic user interface (GUI).

What is claimed is:

1. A server-client network system for a genotyping analysis on a target sample, the server-client network system comprising:
a server including databases required for the genotyping analysis and providing the databases to a client; and
the client receiving the results of a biochip test on the target sample using a biochip and performing the genotyping analysis on the target sample with reference to the databases provided from the server.

2. The server-client network system of claim 1, wherein the databases comprise:
a biochip identifier and layout database storing information on the identifier and layout of the biochip;
an analysis algorithm database storing algorithms required for genotyping analysis; and
a quality control criteria database.

3. The server-client network system of claim 1, wherein the databases stored in the server are built up from statistical data for the results of tests on a number of patient and reference samples using the biochip.

4. The server-client network system of claim 2, wherein the databases stored in the server are built up from statistical data for the results of tests on a number of patient and reference samples using the biochip.

5. The server-client network system of claim 1, wherein the client comprises:
an optical scanning system through which the results of the biochip test on the target sample are received; and
an identifier recognizer which recognizes the identifier of the biochip.

6. The server-client network system of claim 1, wherein the client comprises an engine for performing logical functions including:
a function of detecting the identifier of the biochip;
a function of selecting databases corresponding to the identifier of the biochip;
a function of selecting a database position mode from between a server mode and a local replication mode;
a function of downloading the databases corresponding to the identifier of the biochip from the server if the local replication mode is selected and it is determined that the databases do not exist in the client; and
a function of performing a genotyping analysis on the target sample with reference to the downloaded databases if the local replication mode is selected or performing a genotyping analysis on the target sample with reference to the databases stored in the server if the server mode is selected.

7. The server-client network system of claim 2, wherein the client comprises an engine for performing logical functions including:
a function of detecting the identifier of the biochip;
a function of selecting databases corresponding to the identifier of the biochip;
a function of selecting a database position mode from between a server mode and a local replication mode;
a function of downloading the databases corresponding to the identifier of the biochip from the server if the local replication mode is selected and it is determined that the databases do not exist in the client; and
a function of performing a genotyping analysis on the target sample with reference to the downloaded databases if the local replication mode is selected or performing a genotyping analysis on the target sample with reference to the databases stored in the server if the server mode is selected.

8. The server-client network system of claim 7, wherein the function of performing the genotyping analysis on the target sample comprises:
a function of reading the biochip identifier and layout database;
a function of reading the results of the test on the target sample input via the optical scanning system;
a function of linking the results of the test on the target sample to spot position information stored in the biochip identifier and layout database;
a function of reading the quality control criteria database;
a function of screening out failed spots from among the results of the test based on the quality control criteria database;
a function of reading the analysis algorithm database;
a function of performing a genotyping analysis on the target sample with reference to the analysis algorithm database; and
a function of storing and/or displaying the results of the genotyping analysis.

9. A computer readable medium for a server-client network system for genotyping analysis, the computer readable medium having computer executable instructions for a client to perform logical operations comprising;
receiving databases required for a genotyping analysis from a server; and
performing a genotyping analysis on a target sample using the results of a biochip test on the target sample with reference to the databases.

10. The computer readable medium of claim 9, wherein the databases comprise:
a biochip identifier and layout database storing information on the identifier and layout of the biochip;
an analysis algorithm database storing algorithms required for genotyping analysis; and
a quality control criteria database.

11. The computer readable medium of claim 10, wherein the step of receiving the databases comprises logical steps including:
detecting the identifier of the biochip;
selecting databases corresponding to the identifier of the biochip;
selecting a database position mode from between a server mode and a local replication mode; and
downloading the databases corresponding to the identifier of the biochip from the server if the local replication mode is selected and it is determined that the databases do not exist in the client, and
the step of performing the genotyping analysis comprises logical steps including:
reading the biochip identifier and layout database from among the databases stored in the server if the server mode is selected or reading a biochip identifier and layout database from among the downloaded databases if the local replication mode is selected;
reading the results of the biochip test on the target sample input via an optical scanning system;
linking the results of the biochip test on the target sample to spot position information stored in the biochip identifier and layout database;
reading the quality control criteria database;
screening out failed spots from among the results of the biochip test based on the quality control criteria database;
reading the analysis algorithm database;
performing a genotyping analysis on the target sample based on the analysis algorithm database; and
storing and/or displaying the results of the genotyping analysis.
